(19) United States
(12) Patent Application Publication
(10) Pub. No.: US 2006/0084846 A1
(43) Pub. Date: Apr. 20, 2006

(54) Homeostatic emergent biofeedback representations

(76) Inventor: Ryan Marshall Deluz, Encinitas, CA (US)

Correspondence Address: Ryan Deluz, 963 Urania Avenue, Encinitas, CA, 92024, US

(21) Appl. No.: 10/958,146

(22) Filed: Oct. 4, 2004

(51) Int. Cl. A61B 5/00

(52) U.S. Cl. 600/300; 128/905

(57) ABSTRACT

A method to increase the ease and effectiveness of biofeedback by dynamically representing physiological indicators on a computer display using emergent systems and homeostatic equations. This display shows a number of similar objects that respond both to each other and to changes in physiological measurements. These objects are kept moving and kept within the display's boundaries by using homeostatic equations.

### CROSS-REFERENCE TO RELATED APPLICATIONS

Not Applicable

### FEDERALLY SPONSORED RESEARCH

Not Applicable

### SEQUENCE LISTING OR PROGRAM

A sample program, that is one embodiment of my homeostatic emergent biofeedback representations, is included on CD-ROM as object code containing executable instructions for a computer with a Pentium-type processor running Microsoft® WindowsXP® and having Microsoft® DirectX® version 9.0c or greater and the .NET Framework version 1.0 or greater installed.

### BACKGROUND OF THE INVENTION

### Field of Invention

This invention relates to biofeedback, specifically to the display of representations of measurable physiological indicators using homeostatic equations and emergent systems.

### Biofeedback

This invention relates to biological feedback systems, where an apparatus is used to measure a physiological indicator of a user, and where the corresponding detected signal, or an output responsive thereto, is represented to the user. This enables the user to perceive, for example, his or her heart-rate or brain activity.

This feedback teaches the user to change or control the represented physiological indicator and therefore change or control his or her state. The representation of the detected signal is generally a line-graph, a series of bar-graphs, or a pitch change of an auditory tone such as is recommended in U.S. Pat. No. 3,890,957 to Freeman (1975). In this way, control of the auditory tone, line-graph, or series of bar-graphs represents control of the physiological indicator of the user. This allows the user to learn about his or her current state, and to learn how to change his or her state towards an intended target-state, such as relaxation or focus.

Although this feedback enables users to alter their physiological indicators towards the target-state, users lose interest in line-graph representations and become agitated by pitch-change representations. This is a problem as it often requires forty half-hour sessions of watching line-graphs, or listening to pitch changes, to train the user to easily achieve the target-state.

As the capabilities and processing speed of personal computers increased, inventors created several other computerized representations of physiological indicators. U.S. Pat. No. 6,402,520 to Freer (2002) includes a display of a bug on a leaf that moves more frequently when the user achieves greater focus. U.S. Pat. No. 6,358,201 to Childre, et al. (2002) includes a display of a balloon that flies when the user's heart-rate expresses a desired pattern. In 2003 the Wild Divine Project, at 3330 Eldorado Springs Drive, Boulder, Colo. 80025, released an interactive biofeedback computer game entitled “The Journey to Wild Divine” that contains representations of balls juggling, rocks stacking, doors opening, and rain falling, all of which are linked to the user's physiological indicators.

Although these graphical representations are more engaging to the user than simple line-graphs, difficulties still arise. Because the representations are generally created so that the user experiences the full range of possibilities of the representation within a single session (from a completely closed door to a completely open door), the user tires of the repetition of the same representation over the course of multiple sessions. In addition, users can find such representations frustratingly static if they are unsuccessful for a period of time (the door would remain completely closed and motionless). This frustration can make it more difficult to achieve the target-state, especially if the target-state is relaxation.

To maintain user interest, inventors created systems that allow the user to configure the representation. However, these systems have difficulty displaying complex information, and do not solve the problem of user frustration.

U.S. Pat. No. 6,652,470 to Patton, et al. (2003) describes a method of reducing the symptoms of an individual having attention deficit hyperactive disorder (ADHD) by first obscuring an image and subsequently allowing the user to reduce the level of obscuration by altering a physiological indicator (in this case the user's peripheral skin temperature). Since it is possible to reduce the obscuration of any image, the user can choose an image that he or she likes and that helps in the achievement of the target-state. Nevertheless, watching changes in a single image for a period of time suitable for a biofeedback session (ten to thirty minutes or more), is often not stimulating enough to hold a user's attention, especially a user having an attention deficit.

U.S. Pat. No. 6,450,820 to Palsson, et al. (2002) shows a more suitable method for individuals with ADHD. This patent describes a method and apparatus for providing feedback of the user's physiological indicators using a game of their choice from a wide selection of commercial computer games. The user's physiological indicators are represented by a change in responsiveness of the game input device (e.g. joystick or button control). The target-state used by clinicians when treating individuals with ADHD is a focused, alert state indicated by an increase in higher-frequency brain waves, and a decrease in lower-frequency brain waves. Although this is useful for individuals with ADHD since the user can choose a game he or she is interested in, and that game will demand the user's attention, a significant number of users work to achieve a calm, less hyper-alert, target-state. A video game requiring alertness and speed of response, as well as active muscle movements, is not appropriate for these users. In addition, this method of representation can only give general feedback through the change in responsiveness of the controller. It does not directly display a representation of the physiological measurements, nor does it allow for displays of complex information such as simultaneous display of multiple physiological indicators.

Control of animations, video-clips, and movies is a method used to represent physiological indicators in the Biograph Infinity™ software made by Thought Technology Ltd., 2180 Belgrave Avenue, Montreal, Quebec, Canada, H4A 2L8. This software allows the user or clinician to choose an animation or video-clip that plays forwards when the user is in the target-state and pauses if the user is not in the target-state. Although the clinician can choose a video-clip appropriate to both the user and the target-state, the representation can only be in two possible states, video-clip playing or video-clip not playing. Therefore, the user knows if he or she is in the target-state, or not in the target-state, but does not how close he or she is to achieving the target-state, and does not know if he or she is moving in the direction of achieving the target-state. This makes it more difficult for the user to achieve the target-state and also causes the user frustration. The length of the video-clips can also cause difficulty. Short video-clips must be played repeatedly in order to provide continuous feedback over the duration of one or more sessions. Longer clips, such as movies, often contain narrative elements that can influence the emotional and physiological state, and therefore the physiological indicators, of the user. The narrative elements can also cause frustration when they are paused. In addition, locating appropriate animations or video-clips and loading them into the system can be time consuming and can pose licensing issues on copyrighted media.

In the field of computer animation and special effects, “particle-systems” are often used to simulate natural phenomena such as smoke, grass, clouds, fireworks, or fire. Particle-systems are animated displays of similar objects, such as points, images, two-dimensional shapes, or three-dimensional objects, where each object has properties such as position, velocity, color, and lifetime and where one or more of these properties is random. These properties directly or indirectly effect the behavior of the particle, and/or how the particle is displayed.

Patent pending application Ser. No. 10/867,500, to Ryan Deluz, proposes a configurable particle-system in which a variety of settings can be applied to create a display that is well suited for each client. It also allows clinicians to display precise information on a variety of physiological indicators simultaneously in a manner easily understandable to the user. This method of biofeedback representation, however, still has problems securing user interest, promoting positive response, reacting with a variety of sensitivities, and displaying complex information. Like all other previously patented biofeedback systems, a particle-system displays a direct representation of the user's current state, moving from one fairly static representation to the next. This repetition is boring for the user and provides no unpredictable movements to keep the user genuinely interested. These static qualities can also become frustrating if the user feels that they are not able to change the display and therefore fall further away from their goal of relaxation or focus. Another concern with particle-systems is that the objects in such systems only “live” (show continuously on-screen) for a few seconds, before they “die” (disappear) and are subsequently “born” (appear) at the source of the particle-system (with completely new attributes). Consequently, because of their short lifetime, the particle's behavior is influenced only by the current, and last few seconds of data. This limits the amount of previous data and the complexity of the data that such a system can display. Because of this limitation of the lifetime of the particle, displaying the absolute value of a measurement does not allow much interpretation as to the changes in measurements over extended periods of time (such as prior rates of change, overall patterns of change, complex oscillations of the rates of change, etc.). While an improvement, particle-system biofeedback applications are still born out of the traditional direct representation paradigm and therefore share many of the shortcomings of prior approaches.

### Homeostasis

Webster's 1913 dictionary defines homeostasis as the ability and tendency of certain systems to maintain a relatively constant internal state in spite of changes in external conditions. This ability is achieved by the presence of feedback mechanisms which adjust the state of the system to compensate for changes in the external environment. For instance, the body releases sweat to maintain a constant internal temperature when the external temperature raises beyond a certain level. Homeostasis can be applied to biofeedback representations through the use of programmed homeostatic equations that allow a fixed number of objects on a computer display to interact in complex ways while staying within the visible boundaries of the computer screen.

### Emergent Systems

Emergent systems are another example of biological phenomena that can be computer-simulated and applied to biofeedback applications. A system is said to be emergent when it is made up of several separate entities and yet the overall system has properties which the entities themselves do not have. In other words, an emergent behavior is shown when a number of simple entities operate in an environment, forming more complex behaviors as a collective. A common example of an emergent system is a flock of birds; the individual movements and awareness of each bird creates a meta-system with complex properties beyond the behavior of the birds themselves. As applied to a biofeedback display, an emergent system can be created by making each individual object on a computer screen “aware” of the other objects. For example, an object might be programmed to keep a minimum distance from every other object. By programming equations in which each object is reacting to the behavior of nearby objects, a system is created that is both complex and organic. These emergent properties are fascinating to watch and can reflect the user's state in a complex, lifelike, way.

### Objects and Advantages

As opposed to any of the previously mentioned representations, a biofeedback display governed by homeostatic equations and emergent systems can greatly increase the effectiveness of a biofeedback session by deeply engaging the user in his or her session, displaying both simple and complex data simultaneously, encouraging a positive response from the user, and appealing to the user's subconscious (intuitive) learning processes as well as to his or her conscious (intellectual) learning processes.

Biofeedback displays using homeostatic equations and emergent systems are unique in that they make users feel as if they are interacting with an entity that is a reflection of themselves, rather than simply watching a static, linear, simplified display of their complex state. Because emergent systems and homeostatic equations mirror biological processes, the displays that are created using these techniques have the appearance of being “alive” and are therefore profoundly engaging, and insightful, to watch. The objects on the screen are constantly moving, the resultant system is constantly changing in a dynamic fashion, subtle changes in the user's indicators are amplified, and random and surprising elements are constantly being introduced. This increased movement, complexity, magnification, and surprise draws attention and focus from the user. Like particle-systems, such lifelike biofeedback displays can be configured so that the characteristics and range of each display are well suited for each client's intentions and predilections. However, while particle-systems move from one fixed state to the next, a biofeedback display using homeostatic equations and emergent systems moves from one dynamic state to the next. This dynamism is inherently more compelling. In reality, no user's physiological indicators are ever truly static and such lifelike displays are equipped to interest the user by reflecting his or her true complexity.

The complexity of biofeedback displays using emergent and homeostatic representations is not only more interesting, but more functional and effective as well. Both complex and linear data can be displayed simultaneously in such a system, offering the users multiple levels of information with which to achieve their target-state. When the behavior of the objects in such a system is influenced by a user's physiological input, the user gets to see both the microcosmic, detailed results of his or her input (the objects themselves) as well as the macrocosmic emergent system that has resulted from his or her input. Small changes show as variations within a display and large changes show as an entirely different system. Certain immediately responsive characteristics, such as brightness of the entire system, easily display the current measurement, while properties that take a while to effect the display, such as a property that multiplies the x-axis direction by 1.01 each frame, can express current measurements, long-term changes, and rates of changes of the user's indicators. All of these levels of information help the user reach the target-state, and no other biofeedback representation up to this point has attempted to display this wide range of information in a single, interesting representation. In a line-graph representation, a user can see the changing absolute values of their indicators but would have to further analyze the graph to discern any general patterns in their readings. In particle-system applications, the particles have a very short lifetime and thus are limited in the amount of information they can retain, correlate, and display. Because the objects in the proposed homeostatic emergent biofeedback representation are conserved, they can display current data and the effects of prior data simultaneously.

Another benefit of using homeostatic equations and emergent systems with biofeedback representations is the creation of positive feedback loops that prevent the user from feeling frustrated and therefore not reaching his or her target-state. For example, people using biofeedback for the first time often go through an initial period of frustration. When told to relax, for instance, they often become tense from the effort of “trying” to relax. Then, when viewing the display of their indicators, they become frustrated with the unchanging feedback and begin to try harder. Often, at some point, the user in such a situation becomes so frustrated that he or she gives up trying to achieve the target-state. Ironically, it is often at this moment of giving up that the user begins to finally reach his or her target-state. If the goal of a session is to reach a state of deep relaxation, the harder the user “tries” to reach that state, the more difficult it is to attain. In a case such as this one, a system which gives the appearance of change, even with a static measurement, can help the user to achieve their target-state. A certain amount of false positive feedback, although completely discernable from the target-state, allows the user to feel successful and helps in the achievement of the target-state. A positive feedback loop can expedite the process of reaching the target-state, and help the user bypass any unnecessary struggle. As opposed to displays with two possible states (video playing or video not-playing) biofeedback displays utilizing homeostatic equations and emergent systems have a dynamic nature, allowing for increased ease of initial use and thus solving an important biofeedback problem that has not been addressed before.

Interacting with the dynamic nature of this type of biofeedback system can be very similar to interacting with the natural world because of the complexity and responsiveness of the display. This complexity and responsiveness helps the user utilize his or her full range of learning capabilities. Every person processes the complex information they come across in their daily lives both consciously and subconsciously in order to function properly. Just as someone crossing a street is making both a conscious decision to walk and also subconsciously estimating complex calculus equations in order to avoid being hit, conscious and subconscious learning processes can work together in a biofeedback session to help the user reach his or her target-state. Work with derivatives of GSR has shown that rate of change is a useful indicator for certain physiological activity. In a similar manner, derivatives or other complicated mathematical analyses of physiological data may be more meaningful than the original source data. Because an emergent homeostatic biofeedback system involves an environment complex enough to represent complex data in an intuitive manner, it can express information such as rates of change, patterns of change, length of time in any particular state, and complex combinations of physiological indicators that form specific states. Though the conscious mind cannot interpret all of this information simultaneously, a user's subconscious mind (or more specifically the autonomic nervous system), can calibrate to these subtle changes and use this information to reach the target-state with increased efficiency. Over time this leads to greater learning and control, not only of absolute values, but of complex combinations and patterns of values. When provided with a wide range of simple to complex information, a user has many more tools with which to achieve the complicated and subtle task of changing his or her state.

Accordingly, homeostatic emergent biofeedback representations provide several advantages over any existing representation system by:

(a) maintaining interest in the representation by:
(1) providing a display with objects that comprise systems which are constantly changing and complex
(2) providing a display in which subtle changes are greatly amplified
(3) providing a display in which surprising and widely varied displays occur
(4) providing a display that seems intelligent, alive, and is dynamically interactive

(b) assisting the user to achieve the target-state more easily and more rapidly by:
(1) displaying various levels of complex data about the user's state such as rates of change, patterns of change, etc.
(2) displaying various levels of simple and complex data simultaneously within the system
(3) providing a display in which subtle changes are greatly amplified
(4) providing subtle positive feedback so that the user experiences less frustration
(5) providing data that can be utilized by the user's intuitive, subconscious learning processes
(6) providing data that can be utilized by the user's subconscious and conscious learning processes simultaneously

### SUMMARY

My invention is a method of increasing the efficacy of biofeedback by maintaining user enjoyment and displaying complex data in an intuitive manner by representing one or more of the user's physiological indicators using the properties of a homeostatic emergent biofeedback system.

### DRAWINGS—FIGURES

In the drawings, closely related figures have the same number but different alphabetic suffixes.

FIG. 1 is an illustration of a user watching his or her physiological indicators represented on a computer display. The user's indicators are measured by the biofeedback hardware and the resulting measurements are sent to the computer for processing and display.

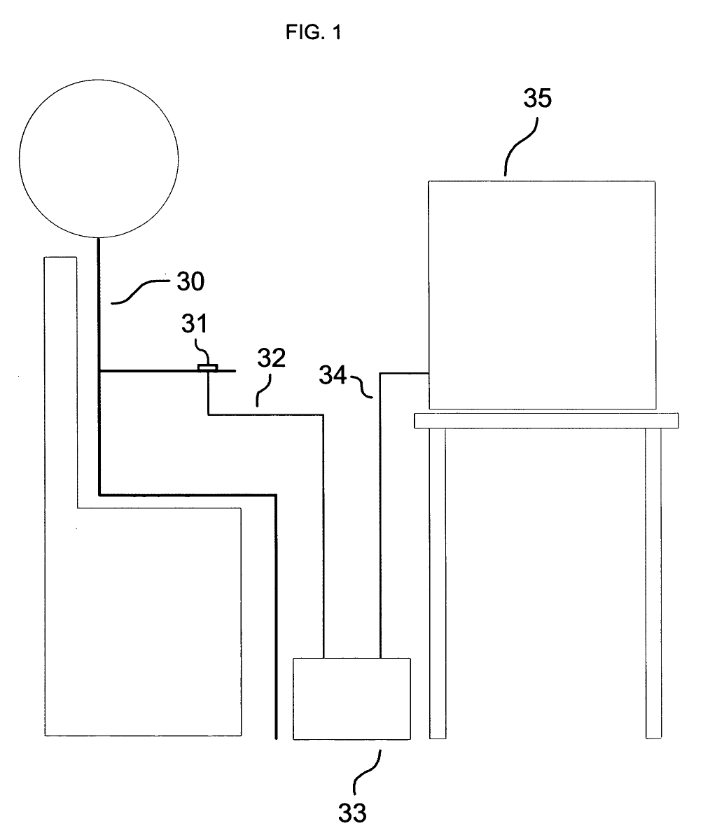

FIG. 2 is an example of a possible interface that links biofeedback data to object properties in a homeostatic emergent biofeedback system.

FIGS. 3A to 3D are illustrations of a possible sequence of simple screenshots showing the movement of objects programmed to move away from each other with only the “Avoid Other Objects” setting.

FIGS. 4A to 4D are illustrations of a possible sequence of simple screenshots showing the movement of objects programmed to move towards the center of the screen with only the “Draw to Center” setting.

FIGS. 5A to 5H are illustrations of a possible sequence of simple screenshots showing the movement of objects programmed with both the “Draw to Center” setting and the “Avoid Other Objects” setting.

FIGS. 6A to 6H are illustrations of a possible sequence of simple screenshots showing the movement of objects programmed with both the “Draw to Center” setting and the “Flock with Objects” setting.

FIGS. 7A to 7H are illustrations of a possible sequence of simple screenshots showing the movement of objects programmed with the “Draw to Center” setting, the “Flock With Objects” setting, and the “Avoid Other Objects” setting.

FIGS. 8A to 8H are illustrations of a possible sequence of simple screenshots showing the movement of objects programmed with the “Form Axes” setting.

FIGS. 9A to 9H are illustrations of a possible sequence of simple screenshots showing the subsequent movement of objects that begin at the location shown in FIG. 8H and that are programmed with the “Only Pairs Can Move” setting.

### DRAWINGS—REFERENCE NUMERALS

30 user

31 biofeedback sensor

32 cable from biofeedback sensor to biofeedback hardware

33 biofeedback hardware

34 cable from biofeedback hardware to computer

35 computer

### DETAILED DESCRIPTION—PREFERRED EMBODIMENT

A preferred embodiment of homeostatic emergent biofeedback representations is as follows:

The embodiment is a computer program written in the programming language C++ that displays a collection of three-dimensional objects on a two-dimensional computer screen. These objects have properties such as location, direction, speed, shape, color, image, and transparency. The computer program uses this information to continuously update and display each object. For instance, the location is continuously updated based on the current direction.

The computer program uses Microsoft® DirectX® to display the objects. DirectX is a set of application programming interfaces (for C++ and other languages) used to provide a low-level hardware interface that speeds the display of three-dimensional data. This allows a complicated representation, such as a complex homeostatic emergent biofeedback system, to be displayed and updated frequently enough to provide the illusion of continuous motion.

The objects' properties, in particular the properties of direction and speed, are controlled by complex equations making the objects' movements appear lifelike. Objects are located in an imaginary space where each object has an x, y, and z location. The x-location of an object is defined by a horizontal line that passes through the center of the screen (the center of the screen is defined as x=0). Objects with a positive x-location are located to the right of the center of the screen, while objects with a negative x-location are located to the left of the center of the screen. The y and z axes are similar to the x-axis, but oriented vertically and depth-wise, respectively.

In this system, if the equation x=x*1.2 acted repeatedly (at the rate of 30 times-per-second) on an object's horizontal location, the object would soon move past the right or left edge of the screen (as long as the initial location of the object was not exactly 0). The equation x=x*1.4 would move the object faster (than x=x*1.2), and the equation x=x/2.0 would move the object towards the center of the screen (towards 0 on the x-axis). When, instead, two equations which are conditional on the location of the object are used, a homeostatic balance is achieved. In a very simple example, one might apply the equation x=x*0.99 if the object is near a horizontal edge of the screen, and x=x*1.01 if the object is near the center of the screen (remember these equations are applied 30 times-per-second).

A more lifelike system can be created by dynamically changing the object's direction instead of the object's location (while still achieving the goal of keeping the objects on the screen). After an object has passed a set maximum distance from the center of the screen this object would slow its movement and then begin to move back towards the center. For instance, if the object's direction were +2.0, (this object moves 2.0 units to the right each time the display is updated) and the object's location was nearing the right edge of the screen, the object's direction is repeatedly decreased (1.9, 1.8, . . . 0 . . . −1.8, −1.9 etc.) until the object's location is closer to the center. This homeostatic process acts on all of the objects simultaneously, and keeps the objects on-screen while creating engaging, dynamic patterns of change. By obeying a simulation similar to the biological rules that provide homeostasis within the body, the objects remain within set parameters of location and form a system that has characteristics of a living entity.

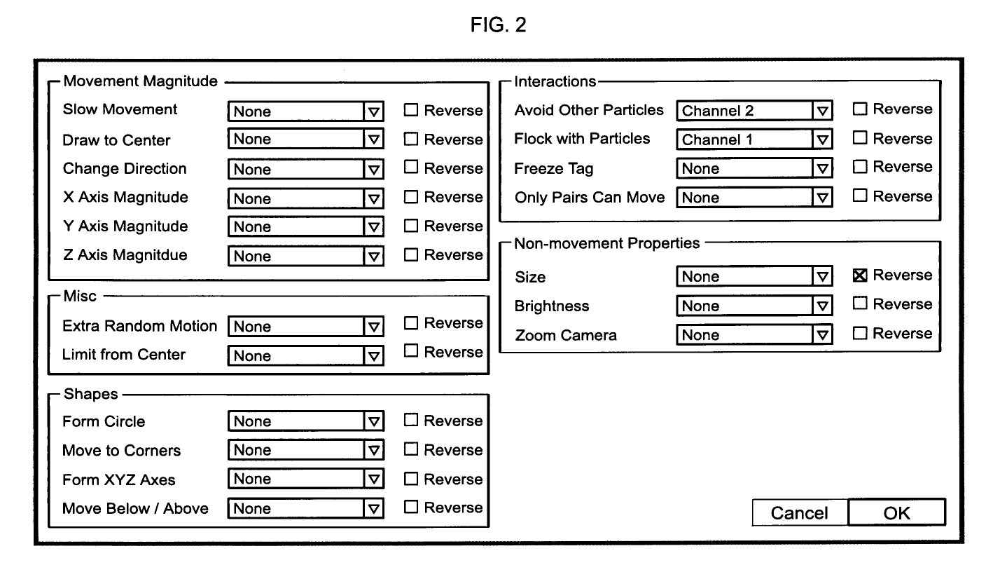

FIG. 2 shows a screenshot of a dialog box allowing various equations to be linked to real-time physiological data from a user. An equation such as “Slow Movement” may act weakly (bringing the object only very slightly more towards the center) or very strongly (quickly bringing the object to the center) depending on the physiological data. A channel number is selected for each equation to be linked. For instance, if the user's heart-rate in channel 1 were linked to the “Slow Movement” property, the speed of the user's heart-rate would control the overall speed of the objects. As the heart-rate increased, the speed would decrease. If, instead, the user wanted the speed to increase as the heart-rate increased, the user would check the “Reverse” box next to the channel selection drop-down.

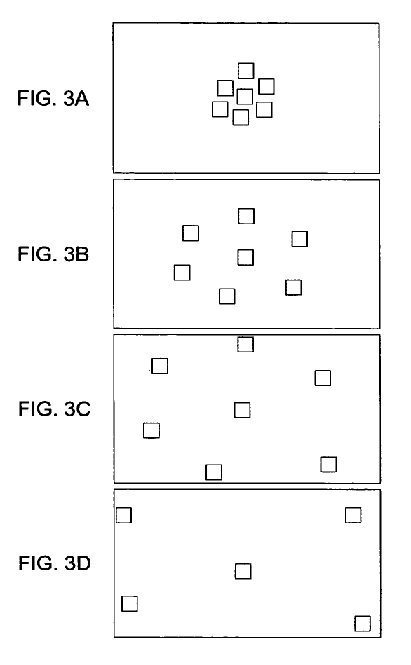

Certain properties, such as “Avoid Other Objects” and “Draw to Center”, are always active, even if not linked to any biological input signal. This allows the display to always be in motion, while keeping the location of the objects within the screen's viewable area. FIGS. 3A to 3D show what would happen if only the “Avoid Other Objects” equation directed the object's movement. The “Avoid Other Objects” equation states that if an object is within a certain minimum distance from any other object, that object's direction is altered so that it moves away from the other object. Once the direction is altered, the object will move continuously in its new direction, until another force acts upon it. Because of this, once an object starts moving in any particular direction, it will soon leave the viewable area of the screen.

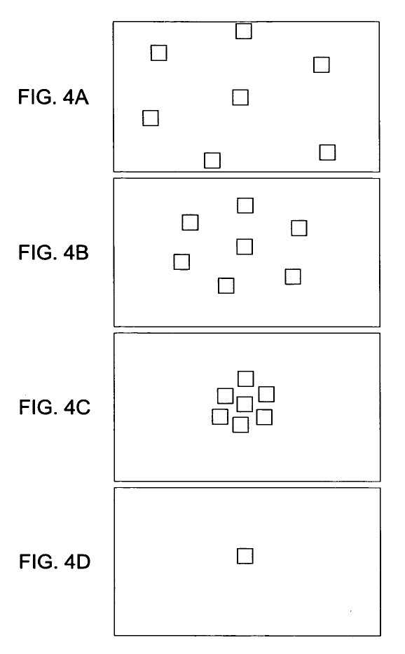

FIGS. 4A to 4D show the property “Draw to Center” which slowly changes the objects' locations and directions so that they move towards the center of the display. If this was the only active force on the objects, all of the objects would soon converge on the exact center of the display. The endpoint of this display, where all of the objects are located at a single point in the center of the screen, is just as uninteresting as the endpoint of the previous display, where all of the objects have moved too far from the center of the screen to be viewable.

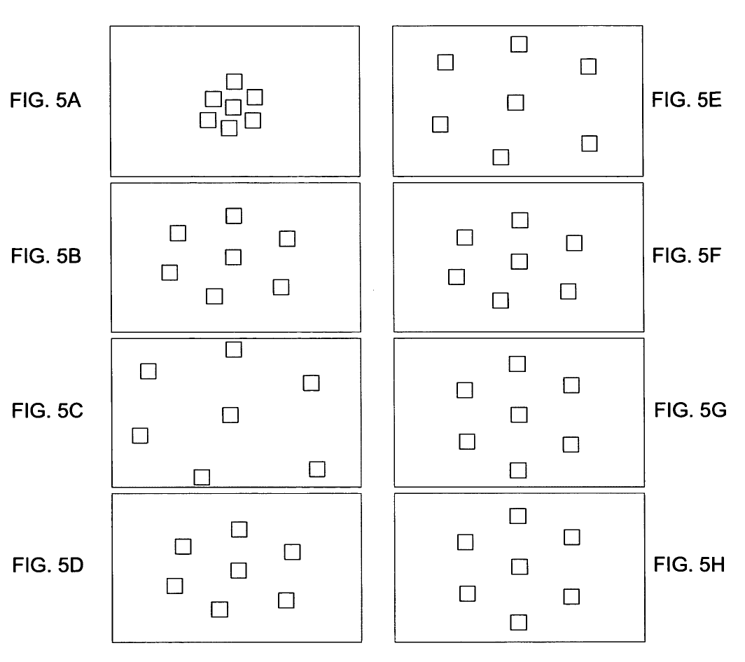

FIGS. 5A to 5H show the properties “Draw to Center” and “Avoid Other Objects” simultaneously directing the objects' movements. In this way the objects dynamically oscillate between moving away from each other and moving back towards the center of the screen, until they finally reach a stable balance point (FIG. 5G and FIG. 5H), where the strength of the “Draw to Center” equation is exactly balanced by the strength of the “Avoid Other Objects” equation.

The strength of these equations can be linked to physiological measurements. Since the display shown is based on the strength of the respective equations, linking physiological measurements to “Draw to Center” and “Avoid Other Objects” allows the display to feedback information about the physiological measurements. One piece of information displayed is the relative strength (ratio) of the two physiological readings. This ratio will set the distance that the objects end up from each other. In addition to the length of time spent at any particular ratio, the dynamic changes between ratios are shown since the system takes a while to reach a stable equilibrium. Even if an equilibrium point is not reached the current distance and directionality of the objects' movements show the current and prior ratios. Since these equations only change the direction incrementally, but do not set an absolute direction or location, much information about prior states is present in the current display, along with information about the current state. However, the current state is most clearly visible, as the largest changes in the display are based on the current equations.

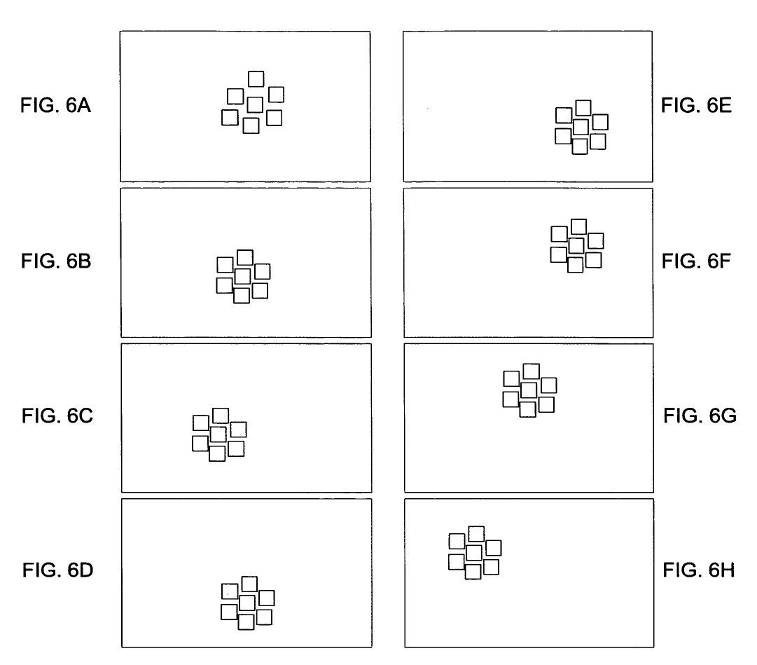

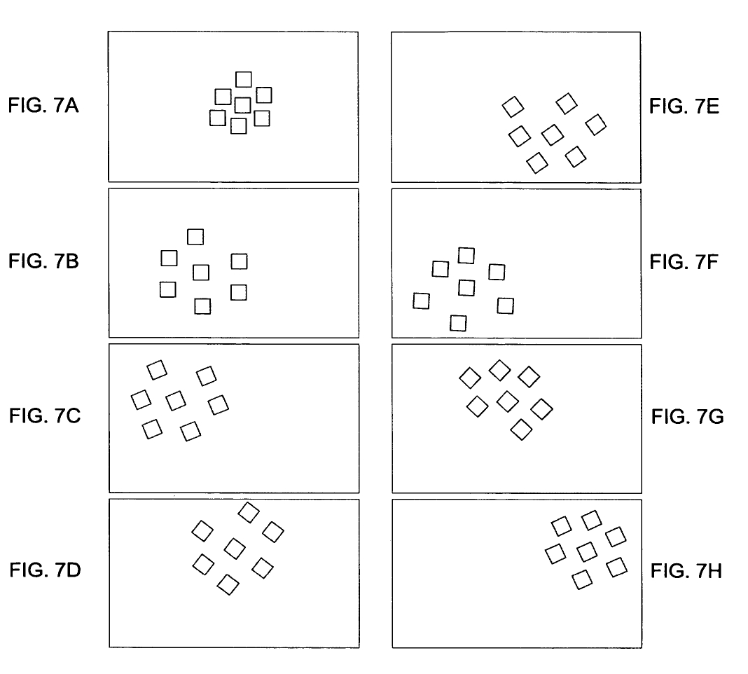

Equations such as “Avoid Other Objects” or “Flock with Objects” produce object-to-object interactions, exemplifying emergent behaviors. Since each object's direction (or other attributes) becomes linked to nearby objects' directions (or other attributes), fascinating, complex, and lifelike behaviors emerge. For instance, although “Flock with Objects” (FIGS. 6A to 6H), “Avoid Other Objects” (FIGS. 3A to 3D), or “Draw to Center” (FIGS. 4A to 4D) do not produce lifelike displays by themselves, when these three properties are combined the resulting display looks very much like a flock of birds circling a center point. A flock of birds flying can act as if they are governed by the simple rules “fly near the flock, and in the same direction as the flock” and “don't come too near, or hit the other members of the flock”. These rules are exactly what is being simulated with the objects on the screen. The simulation is then influenced by the current physiological measurements of the user, allowing the user to control the basic equations governing the movement and interactions of an entire system of unique objects. This is very interesting to observe, and allows the user to experience a feeling of greater control than if he or she were simply moving a line-graph, or starting and stopping a video-clip. FIGS. 7A-7H give a very simple example of movements generated by these three simultaneous directives. In the actual simulated display, the objects would be moving in three-dimensions, and the strengths of the individual properties of flocking and avoiding would be very easy to observe.

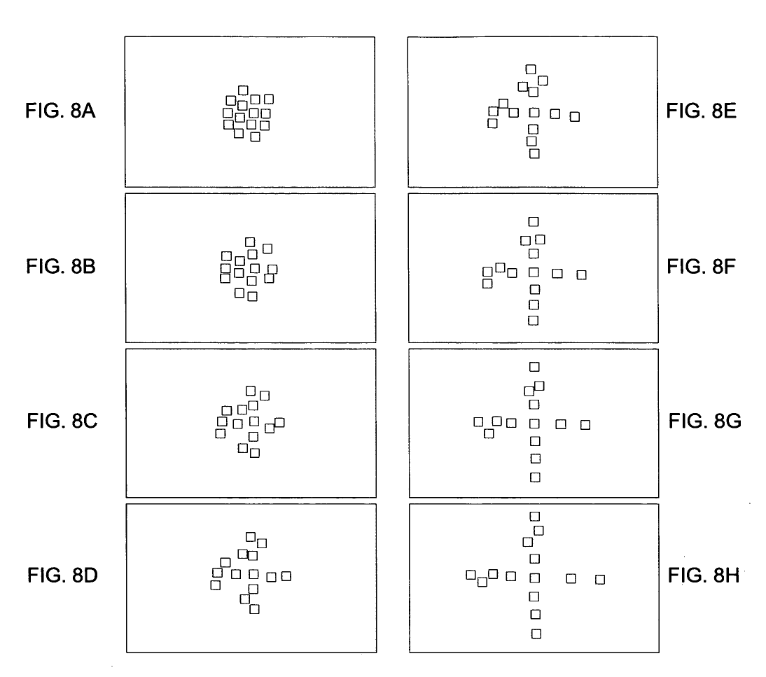

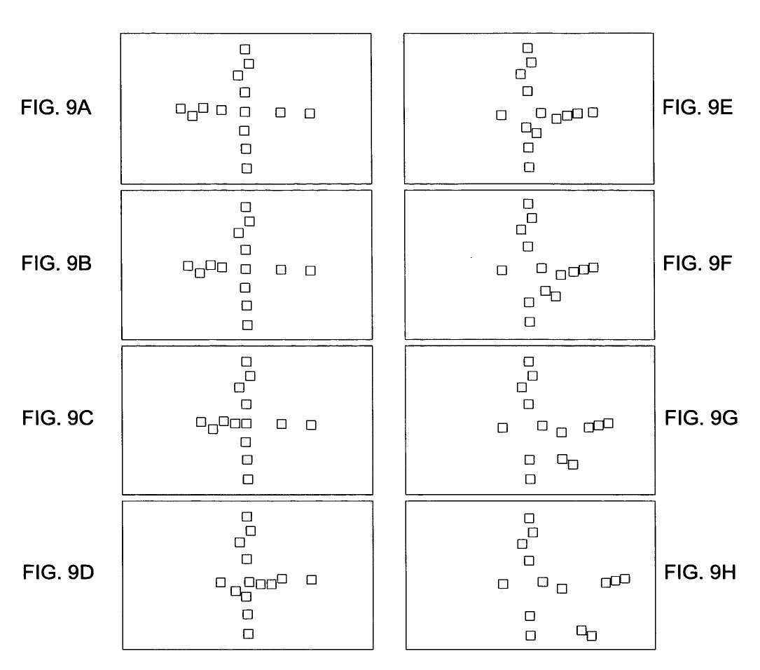

There are many other properties that can be set, as shown in FIG. 2. FIGS. 8A to 8H and FIGS. 9A to 9H show two specific properties in action. FIGS. 8A to 8H show a two-dimensional representation of “Form XYZ Axes”. “Form XYZ Axes” programs the objects to move towards the axis closest to them. A small amount of “Avoid Other Objects” and “Draw to Center” are also present, stopping the objects from colliding with each other, and keeping them viewable on the screen. FIGS. 9A to 9H show the property “Only Pairs Can Move” which only allows objects to move if they are very close to another object. If an object is not very near any other object, it is frozen in space until a moving pair comes near enough to allow it to move.

FIGS. 9A to 9H begin where FIG. 8H ends, showing the result of a sudden change in the strength of two equations. This could under the following circumstances. A user may be using the display to help increase his or her peripheral skin temperature and help relax a specific muscle. Peripheral skin temperature increase can be a sign of relaxation, since a fight-or-flight response causes blood to flow away from the periphery of the body towards the internal organs and brain. For this reason the skin temperature and muscle relaxation often change in a correlated fashion. The muscle tension can be linked to “Form XYZ Axes” so that the axes form when the muscle is tensed. The skin temperature can be linked to “Only Pairs Can Move” so that all objects not near another object freeze when the skin temperature increases. A quick decrease in muscle tension, accompanied by an increase in skin temperature, produces the display shown in FIGS. 9A to 9H. “Form XYZ Axes”, which was previously very strong, becomes weak, while “Only Pairs Can Move”, which was very weak, becomes strong. As “Only Pairs Can Move” dominates the movement of the objects, objects that are not near to another object freeze, while objects that are near, begin to move. In this way, the shift from one state to another can be achieved by changing the representation from a system of objects forming an axes, into a system of objects flying around together. This change in the user's state, caused by the dynamic change of many complex interrelated homeostatic systems within the user's body, is reflected by a dynamic change in the homeostatic emergent behaviors of the objects and system of objects shown on the screen. It is, in fact, a large change for a user to shift from tension to relaxation, and the magnitude of that change is reflected by a fundamental change in the properties and interactions of the objects, producing a large-scale change in the display.

If the user used these same settings over the course of multiple sessions, the display would look similar each time the user relaxed, but would also be unique each time. This display would be different owing to the different speeds of changes and absolute values of the user's physiological indicators. The display would, in fact, be unique even with identical readings, since the objects start in random locations and with random directions. This randomness keeps the display interesting for repeat users.

With the 19 settings shown in FIG. 2, hundreds of different displays with different fundamental equation combinations can be generated. Most possible configurations can easily display large, broad changes (such as the transition from primarily forming axes, to primarily moving only in pairs) as well as very detailed small changes (such as increasing in brightness, avoiding other objects a little more, or slowing movement down a little). These displays can be configured to be appropriate for a user's particular state change, so that the change in the interactions of the objects seems similar to the change in the state of the user (for instance, the objects flocking, or slowing down, when the user becomes calm).

More complex representations are obviously possible to construct. For instance, if “Size”, “Brightness”, “Form XYZ Axes”, and “Slow Movement” are linked to channel 1, “Avoid Other Objects”, and “Flock With Objects” are linked to channel 2 (with the “Reverse” checkboxes checked), and “Only Pairs Can Move” is linked to channel 3, the user can create a great variety of different representations as he or she changes the physiological indicators linked to channels 1, 2, and 3.

Advantages

From the description above, a number of advantages of my homeostatic emergent biofeedback representations become apparent:

(a) A great variety of representations can be created by changing the properties of the objects.

(b) These representations are more similar to the actual changes occurring in the body than any other previous biofeedback representation.

(c) A user using the same settings many times would never see the exact same display twice.

(d) The display can interest users based on the constant movement, lifelike movements, and novelty of the great variety of representations as well as the great diversity possible within any particular configuration of settings.

(e) Users are less frustrated because the display remains interesting even when their physiological measurements are unchanging.

(f) Users can achieve their target physiological measurements more easily, as they are less frustrated by the representation.

(g) Users can achieve their target physiological measurements more easily, as the display feeds back both large changes and very specific changes, making it easy for the users to see if they are heading in the correct direction while also showing any large changes that occurred in their state.

(h) Users rapidly learn how to change the display because the current readings, previous readings, and rates of changes of the readings, are all visible within the system.

Conclusion, Ramifications, and Scope

Accordingly, the reader will see that using homeostatic equations and emergent systems to create biofeedback representations makes it easier for the user to achieve the goal of changing his or her physiological measurements and therefore changing his or her state.

Furthermore, using homeostatic equations with emergent systems in biofeedback representations has the following benefits:

within each set of settings, there are many different shapes and movements that can appear in response to changes in the user's physiological measurements

the displays are lifelike and therefore draw the user's attention

the displays are lifelike and therefore are enjoyable to interact with

the displays can show information about large changes and small changes simultaneously

the displays can show complex information about the ratios, rates of change, and length of time in any particular state, all in one single display

the displays show information about current measurements and previous measurements simultaneously and intuitively

the displays are interesting even when the user's measurements are static, thereby keeping the user not only interested, but also preventing the user from becoming frustrated with an inability to change his or her measurements

the combination of lack of frustration, along with the interesting and easy to understand feedback, assists the user in achieving the goal of changing his or her physiological indicators and associated state.

Although the description above contains many specificities, these should not be construed as limiting the scope of the invention, but as merely providing examples of some of the presently preferred embodiments of this invention. For example, this program could be programmed in another programming language such as Java® instead of C++; the system of objects could be displayed using OpenGL® instead of DirectX®; the system of objects could be displayed as a landscape where the objects are points defining the heights of the landscape; the equations controlling the objects' motion could effect the objects' locations instead of directions, etc.

Thus the scope of the invention should be determined by the appended claims and their legal equivalents, rather than by the examples given.

I claim:

1. A method of representing one or more physiological indicators comprising:
(a) providing a computer with a display
(b) providing a user of said computer
(c) providing one or more physiological indicators of said user
(d) providing an apparatus used to measure said physiological indicators of said user
(e) providing a detected signal measured by said apparatus
(f) providing data stored in said computer where said data is from the group of location, direction, speed, and color
(g) providing objects displayed on said computer wherein said objects have properties comprising of said data
(h) providing a plurality of said objects
(i) providing first instructions for said computer to display said plurality of said objects
(j) providing second instructions for said computer to update said objects' properties
(k) first means for updating the properties of each individual object from said plurality of said objects based on properties of one or more other objects from said plurality of said objects
(l) second means for altering said first means based on said detected signal
whereby said user is presented with a display of said physiological indicators, wherein the display represents the physiological indicators through the interactions of said objects with each other.

2. The second instructions of claim 1 wherein the updating uses equations that keep the said plurality of said objects close enough to the center of the display so as to keep them viewable.

3. The keeping said plurality of said objects close enough to the center of the display of claim 2 wherein said keeping is done by altering the locations of said objects.

4. The keeping said plurality of said objects close enough to the center of the display of claim 2 wherein said keeping is done by altering the directions of said objects.

5. The second instructions of claim 1 wherein the updating uses equations that keep the said plurality of said objects far enough from the center of the display so as to keep them from all converging to the center of the display.

6. The first means of claim 1 wherein the updating is based on the objects' distances from each other.

7. The first means of claim 1 wherein the updating moves each object of said plurality of objects away from other objects of said plurality of objects if the objects are within a certain distance from each other.

8. The first means of claim 1 wherein the updating moves each object of said plurality of objects towards one or more nearby objects.

9. The first means of claim 1 wherein the updating creates an emergent shape for the plurality of objects.

10. The shape of claim 9 wherein the shape is constantly changing.

11. The first means of claim 1 wherein the updating creates an oscillating emergent movement for said plurality of objects.

12. The plurality of said objects of claim 1 wherein the space between the objects is filled in, thereby allowing said objects to define a three-dimensional solid.

13. The second means of claim 1 wherein said detected signal alters the strength of equations controlling the first means.

14. The second means of claim 1 wherein two said physiological indicators alter two properties of said plurality of said objects, such that said objects' properties show a ratio between the two said physiological indicators.

15. The second means of claim 1 wherein said second means are configured using graphical user-interface elements.

16. A method of representing one or more physiological indicators comprising:
(a) providing a user
(b) providing one or more physiological indicators of said user
(c) providing a computational system with a display
(d) providing objects displayed on said computational system
(e) providing a plurality of said objects
(f) providing properties of said plurality of said objects from the group of size, shape, color, image, transparency, location, direction, and speed
(g) providing a device to measure said physiological indicators of said user and transmit said physiological indicators to said computational system
(h) providing instructions for said computational system to change the properties of said plurality of said objects
(i) first means for each said object of said plurality of said objects to respond to the said properties of other said objects of said plurality of said objects
(j) second means for said physiological indicators to alter said first means
whereby said user can change the properties of said-objects, and the emergent properties of said plurality of said objects by changing said physiological indicators.

17. The first means of claim 16 wherein each said object of said plurality of said objects only responds to the properties of nearby objects of said plurality of said objects.

18. The first means of claim 16 wherein each said object of said plurality of said objects responds to the properties of every other said object of said plurality of said objects.

19. The instructions of claim 16 wherein the properties of said plurality of said objects are changed so as to keep said objects visible on the display.

20. The keeping said objects visible of claim 19 wherein said objects are kept visible by changing their locations so that they are closer to the center of the display.

21. The keeping said objects visible of claim 19 wherein the objects are kept visible by changing their directions so that they are increasingly towards the center of the display.

22. The keeping said objects visible of claim 19 wherein said objects are kept from converging in the center of the display by changing their directions so that they move away from each other.

23. The second means of claim 16 wherein said physiological indicators alter the strength of equations that control the direction, speed, color, or other properties of said objects.

24. The second means of claim 16 wherein said second means are configured using graphical user-interface elements.
